(19) United States
(12) Patent Application Publication
(10) Pub. No.: US 2026/0290045 A1
(43) Pub. Date: Sep. 24, 2026

(54) INFORMATION PROCESSING DEVICE, INFORMATION PROCESSING SYSTEM, INFORMATION PROCESSING METHOD, AND NON-TRANSITORY STORAGE MEDIUM

(71) Applicant: HONDA MOTOR CO., LTD., Tokyo (JP)

(72) Inventors: Yoko Umeyama, Tokyo (JP); Tetsuya Gomi, Tokyo (JP); Masahiro Kimura, Wako-shi (JP); Taiki Iwama, Tokyo (JP); Ryuji Iwashita, Wako-shi (JP); Seungho Choi, Wako-shi (JP); Kotaro Masuda, Wako-shi (JP); Yuji Yasui, Wako-shi (JP); Jing Zhao, Tokyo (JP); Adil Gupta, Tokyo (JP); Ryo Takebayashi, Tokyo (JP)

(21) Appl. No.: 19/551,679

(22) Filed: Feb. 27, 2026

(30) Foreign Application Priority Data
Mar. 24, 2025 (JP) 2025-048550

(51) Int. Cl. G06V 20/59; B60Q 9/00; G06V 10/62; G06V 20/58; G06V 40/16; G06V 40/18

(52) U.S. Cl. CPC G06V 20/597; B60Q 9/00; G06V 10/62; G06V 20/58; G06V 40/171; G06V 40/193

(57) ABSTRACT

An information processing device acquires face images including a face of a driver of a vehicle in a time series; extracts feature quantities of the face of the driver on the basis of the face images; and determines whether or not an abnormality has occurred in the driver on the basis of a change tendency of the feature quantities.

### CROSS-REFERENCE TO RELATED APPLICATION

Priority is claimed on Japanese Patent Application No. 2025-048550, filed Mar. 24, 2025, the content of which is incorporated herein by reference.

### BACKGROUND

### Field of the Invention

The present invention relates to an information processing device, an information processing system, an information processing method, and a non-transitory storage medium.

### Description of Related Art

In recent years, efforts have been active to provide access to sustainable transportation systems that take into account vulnerable road users such as the elderly, persons with disabilities, and children among traffic participants. Toward realizing this, research and development efforts are being focused on further improving traffic safety and convenience, particularly through the development of mobility means for the elderly and persons with disabilities. (Japanese Unexamined Patent Application, First Publication Nos. 2020-71528 and 2017-16568)

### SUMMARY

Incidentally, in a case in which dementia develops, there is a tendency for abrupt changes in emotion to occur frequently, as well as a tendency for emotions to show little change even in dangerous situations (apathetic). A driver who has developed dementia may exhibit these tendencies even while driving. If a driver continues to drive without being aware of the onset of dementia, there is concern that traffic safety may be compromised, and thus it is important to routinely monitor the driver's emotions (facial expressions) and to detect and notify a decline in the cognitive ability at an early stage.

The present invention is in consideration of such situations, and one object thereof is to provide an information processing device, an information processing system, an information processing method, and a non-transitory storage medium capable of detecting driver's abnormalities, and, furthermore, it contributes to the development of a sustainable transportation system.

An information processing device, an information processing system, an information processing method, and a non-transitory storage medium according to the present invention employ the following configurations.
(1): According to one aspect of the present invention, there is provided an information processing device including: a storage medium storing computer-readable instructions; and one or more processors connected to the storage medium, the one or more processors executing the computer-readable instructions to: acquire face images including a face of a driver of a vehicle in a time series; extract feature quantities of the face of the driver on the basis of the face images; and determine whether or not an abnormality has occurred in the driver on the basis of a change tendency of the feature quantities.
(2): In the aspect (1) described above, the one or more processors execute the computer-readable instructions to extract the feature quantities of the face of the driver in each of the face images acquired in the time series.
(3): In the aspect (2) described above, the one or more processors execute the computer-readable instructions to: derive an amount of change between a first feature quantity and a second feature quantity, the first feature quantity being extracted from the face image acquired at a first time point, the second feature quantity extracted from the face image acquired at a second time point, the second time point being earlier than the first time point; and determine whether or not an abnormality has occurred in the driver on the basis of the amount of change.
(4): In the aspect (3) described above, the one or more processors execute the computer-readable instructions to: calculate the amount of change between sets of two face images being consecutive in time in the face images acquired in the time series; and determine whether or not an abnormality has occurred in the driver on the basis of the number of the amounts of change being a threshold or more among a plurality of the calculated amounts of change.
(5): In the aspect (4) described above, the one or more processors execute the computer-readable instructions to calculate the amount of change for each of the sets of two consecutive face images.
(6): In any one of the aspects (3) to (5) described above, the one or more processors execute the computer-readable instructions to: further acquire a surrounding image of the vehicle; detect presence or absence of a risk object in the surrounding image; and determine that an abnormality has occurred in the driver in a case in which the risk object has not been detected, and in a case in which the amount of change corresponding to a time point at which the surrounding image has been acquired is a threshold or more.
(7): In any one of the aspects (3) to (5) described above, the one or more processors execute the computer-readable instructions to: further acquire a surrounding image of the vehicle; detect presence or absence of a risk object in the surrounding image; and determine that an abnormality has occurred in the driver in a case in which the risk object has been detected, and in a case in which the amount of change corresponding to a time point at which the surrounding image has been acquired is less than a threshold.
(8): In any one of the aspects (1) to (7) described above, the one or more processors execute the computer-readable instructions to cause a notification device to give a notification in a case in which it is determined that an abnormality has occurred in the driver.
(9): According to one aspect of the present invention, there is provided an information processing system including: the information processing device according to any one of the aspects (1) to (8) described above; a first terminal device used by the driver; and a second terminal device used in a medical institution, the information processing device causing: the first terminal device to perform content recommending to receive a medical treatment of the medical institution in a case in which it is determined that an abnormality has occurred in the driver; and the second terminal device to give a notification on the basis of information indicating an intention to accept the medical treatment input to the first terminal device by the driver.
(10): According to one aspect of the present invention, there is provided an information processing system including: the information processing device according to any one of the aspects (1) to (8) described above; a first terminal device used by the driver; and a second terminal device used by a user different from the driver, the second terminal device having notifications controlled by the information processing device and issuing a notification to the first terminal device on the basis of information input in accordance with a user's operation.
(11): According to one aspect of the present invention, there is provided an information processing method using a computer, the information processing method including: acquiring face images including a face of a driver of a vehicle in a time series; extracting feature quantities of the face of the driver on the basis of the face images; and determining whether or not an abnormality has occurred in the driver on the basis of a change tendency of the feature quantities.
(12): According to one aspect of the present invention, there is provided a non-transitory storage medium having a program stored thereon that causes a computer to: acquire face images including a face of a driver of a vehicle in a time series; extract feature quantities of the face of the driver on the basis of the face images; and determine whether or not an abnormality has occurred in the driver on the basis of a change tendency of the feature quantities.

According to the above-described aspects (1) to (12) of the present invention, a decline in the driver's cognitive ability can be detected.

### BRIEF DESCRIPTION OF THE DRAWINGS

FIG. 1 is a diagram showing an example of the configuration and the use environment of a driving monitoring device including an information processing device according to a first embodiment.

FIG. 2 is a diagram showing an example of a driving monitoring device according to the first embodiment.

FIG. 3 is a diagram showing an example of an image acquired by the driving monitoring device according to the first embodiment.

FIG. 4 is a diagram for describing an oversight warning process performed by the driving monitoring device according to the first embodiment.

FIG. 5 is a diagram showing an example of information detected by a detector according to the first embodiment.

FIG. 6 is a diagram showing an example of a warning issued by a warning part according to the first embodiment.

FIG. 7 is a diagram for describing an abnormality notification process performed by the driving monitoring device according to the first embodiment.

FIG. 8 is a diagram showing an example of amount-of-change information according to the first embodiment.

FIG. 9 is a diagram showing an example of a relationship between the amount of change and a frequency according to the first embodiment.

FIG. 10 is a flowchart showing an example of the flow of a process executed by an information processing device according to the first embodiment.

FIG. 11 is a diagram showing an example of the flow of a process executed by the information processing device according to the first embodiment.

FIG. 12 is a diagram for describing an abnormality notification process of an information processing system according to a second embodiment.

### DETAILED DESCRIPTION OF THE INVENTION

Hereinafter, an information processing device, an information processing system, an information processing method, and a non-transitory storage medium according to embodiments of the present invention are described with reference to the drawings. In the following description, a forward direction of a vehicle is described as a positive X direction, a backward direction of the vehicle is described as a negative X direction, a direction that is a widthwise direction of the vehicle and is toward the right side with reference to the positive X direction is described as a positive Y direction, a leftward direction is described as a negative Y direction, and a height direction of the vehicle that is a direction orthogonal to the X direction and the Y direction is described as a positive Z direction.

### First Embodiment

### [Whole Configuration]

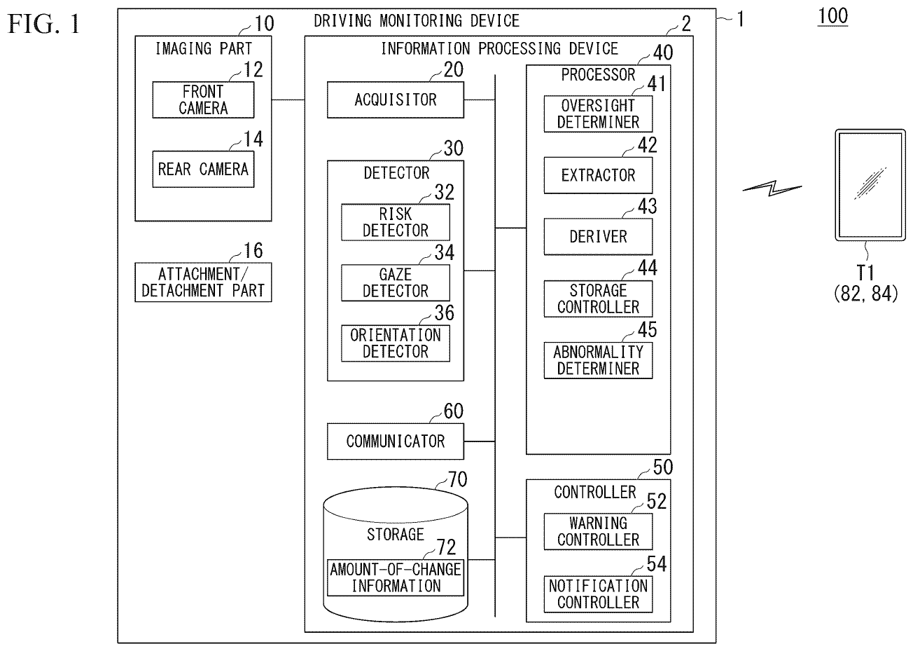

FIG. 1 is a diagram showing an example of the configuration and the use environment of a driving monitoring device 1 including an information processing device 2 according to a first embodiment. The information processing device 2, for example, is mounted in a driving monitoring device 1 that is retrofitted to a vehicle (hereinafter referred to as “vehicle M”) such as a drive recorder. In other words, the driving monitoring device 1 (information processing device 2) is attachable to and detachable from the vehicle M via an attachment/detachment part (hereinafter referred to as an “attachment/detachment part 16”). The information processing device 2 is, for example, a device that is not connected to an in-vehicle network of the vehicle M. The in-vehicle network is an in-vehicle network that uses communication standards of a Controller Area Network (CAN), to which Electronic Control Units (ECUs) relating to vehicle control are connected, including driving, braking, steering, various vehicle sensors, drive operators, operation buttons, and the like of the vehicle M. A network of the vehicle M for a Human Machine Interface (HMI) may be excluded from the in-vehicle network described above.

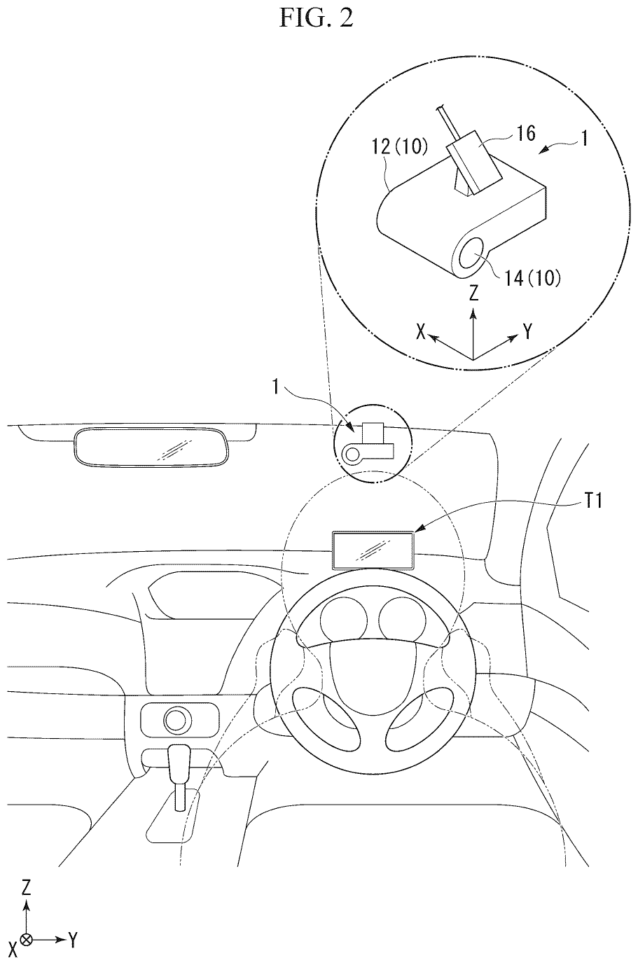

FIG. 2 is a diagram showing an example of the driving monitoring device 1. The driving monitoring device 1 is mounted, for example, at a portion that is near the front windshield of the vehicle M and positioned in the vicinity of a side in front of a driver's seat. The driving monitoring device 1 stores images (moving images) captured by the imaging part 10, for example, in a storage 70 (described below). The driving monitoring device 1, for example, may be configured to include a display device such as a Liquid Crystal Display (LCD) and to be able to display images (moving images) stored in the storage 70 on a display device.

The driving monitoring device 1 includes, for example, an imaging part 10, an attachment/detachment part 16, and an information processing device 2. In the shown example, the imaging part 10 includes a front camera 12 and a rear camera 14.

Each of the front camera 12 and the rear camera 14 is, for example, a digital camera that uses a solid-state imaging element such as a Charge Coupled Device (CCD) or a Complementary Metal Oxide Semiconductor (CMOS). The front camera 12 repeatedly (periodically) images the surroundings in front of the vehicle M from a position at which the driving monitoring device 1 is attached. The rear camera 14 repeatedly (periodically) images the surroundings to the rear of the vehicle M and the interior of the vehicle M from a position at which the driving monitoring device 1 is attached. The horizontal field of view of each of the front camera 12 and the rear camera 14 is, for example, 180 degrees or more. In other words, the driving monitoring device 1 (the imaging part 10) images a 360-degree range around the vehicle M from the attached position. Each of the front camera 12 and the rear camera 14 may be, for example, a fisheye camera including a fisheye lens. Alternatively, each of the front camera 12 and the rear camera 14 may be a wide-angle camera or the like. In the present embodiment, although the front camera 12 and the rear camera 14 are described as being used, instead of or in addition to these, other cameras may be used. In other words, the imaging part 10 may be configured such that images used in the present embodiment (surrounding images and face images described below) are captured.

The attachment/detachment part 16 is, for example, a member used for mounting the driving monitoring device 1 on the vehicle M. The attachment/detachment part 16, for example, is an arbitrary member such as a supporting member including a suction cup, an adhesive sheet, a bracket, and the like. The attachment/detachment part 16 may also include a power supply cable (a wiring or the like) for supplying power to the driving monitoring device 1.

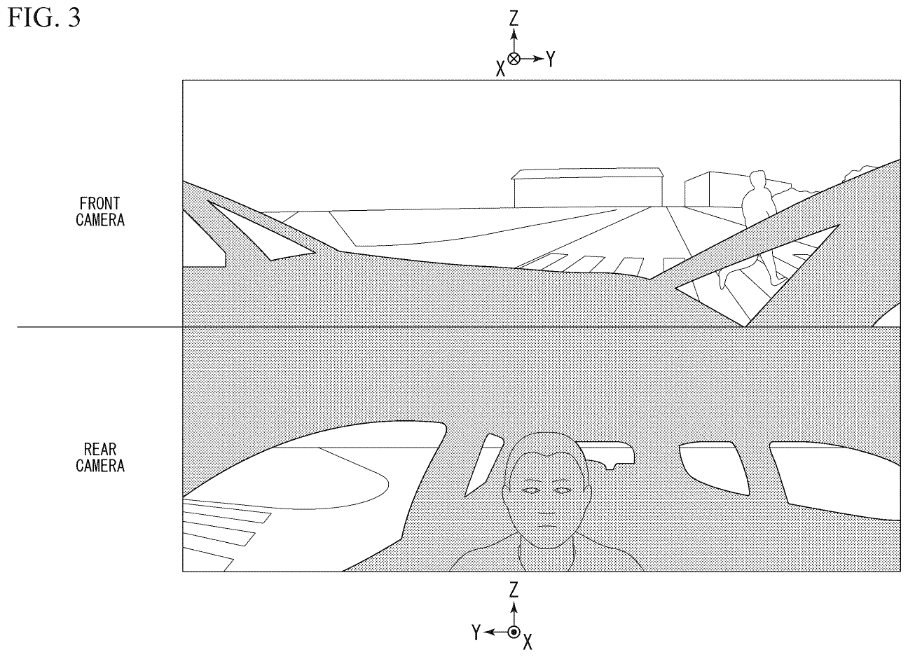

FIG. 3 is a diagram showing an example of an image acquired by the driving monitoring device 1. In other words, FIG. 3 is a diagram showing an example of an image captured by the imaging part 10. The front camera 12 images a side in front of the vehicle M through the front windshield, the driver-side window, and the passenger-side window. The rear camera 14 images the interior of the vehicle M (occupants including the driver), a side to the rear of the vehicle M through the driver-side window, the passenger-side window, and the rear window, and the like.

In the image captured by the front camera 12 (hereinafter referred to as a “front image”), as subjects, for example, objects such as other vehicles, pedestrians, bicycles, fixed objects, road lane markings, and the like that are objects present in front of the vehicle M as seen through the front windshield and objects present on the left and right sides (forward directions) of the vehicle M as seen through the left and right side windows are drawn.

In the image captured by the rear camera 14 (hereinafter referred to as a “rear image”), objects present in the interior of the vehicle M, objects present on a side to the rear of the vehicle M as seen through the rear window, and objects present on the left and right sides (rearward direction) of the vehicle M as seen through the left and right side windows are drawn. For this reason, in the rear image captured by the rear camera 14, as s subject, at least a driver of the vehicle M to which the driving monitoring device 1 is attached is also drawn.

The front image and the rear image include images acquired by imaging the surroundings of the vehicle M (hereinafter referred to as “surrounding images”). The rear image includes an image acquired by imaging the face of the driver of the vehicle M (hereinafter referred to as a “face image”). The driving monitoring device 1 may be attached to any location, as long as it is a position at which the surrounding image and the face image can be captured by the imaging part 10.

The information processing device 2 (see FIG. 1) causes a warning and a notification to be output through the first terminal device T1 on the basis of results of referring to a surrounding image and a face image output by the imaging part 10. The driving monitoring device 1 and the first terminal device T1 may together be referred to as an “information processing system 100”.

The first terminal device T1 is, for example, a portable terminal device such as a smartphone or a tablet terminal that is used by a driver who drives the vehicle M to which the driving monitoring device 1 is attached. In the first terminal device T1, for example, an application for receiving warnings and notifications from the information processing device 2 is executed. The application causes a display device to display an image based on information transmitted by the information processing device 2 (for example, warning information and notification information described below) or causes a speaker to output a sound. The first terminal device T1 is also referred to as a warning part 82 and a notification part 84. The first terminal device T1 is used, for example, in the state of being detachably attached to the vehicle M. For example, a holder for the first terminal device T1 that has an attachment/detachment part is provided in one or both of the first terminal device T1 and the vehicle M, and the first terminal device T1 is supported by the holder.

In place of the first terminal device T1, a navigation system of the vehicle M, a display, or a speaker may be used. In other words, the navigation system of the vehicle M, the display, or the speaker may output warnings and notifications on the basis of instructions from the information processing device 2. In other words, the navigation system of the vehicle M or the display or the speaker of the vehicle M, may be configured as the warning part 82 or the notification part 84. In place of the first terminal device T1, the driving monitoring device 1 may be provided with a display (the warning part 82, the notification part 84), a speaker (the warning part 82, the notification part 84), or the like. The notification part 84 is an example of a “notification device” in the claims.

As shown in FIG. 1, the information processing device 2 includes, for example, an acquisitor 20, a detector 30, a processor 40, a controller 50, a communicator 60, and a storage 70. The detector 30 includes a risk detector 32, a gaze detector 34, and a face orientation detector 36. The processor 40 includes an oversight determiner 41, an extractor 42, a deriver 43, a storage controller 44, and an abnormality determiner 45. The controller 50 includes a warning controller 52 and a notification controller 54.

Each of the acquisitor 20, the detector 30, the processor 40, and the controller 50, for example, includes a hardware processor such as a central processing unit (CPU) and a storage device (a storage device including a non-transitory storage medium) storing a program (software), and the function of each constituent element is realized by the processor executing the program. Some or all of such constituent elements may be realized by hardware (a circuit part; including circuitry) such as a Large Scale Integration (LSI), an Application Specific Integrated Circuit (ASIC), a Field-Programmable Gate Array (FPGA), or a Graphics Processing Unit (GPU), or the function of each constituent element may be realized by software and hardware in cooperation. For some or all of such constituent elements, the function of each of the constituent elements may be realized by a dedicated LSI.

The program (software) may be stored in advance in a storage 70 (a storage device including a non-transitory storage medium) included in the information processing device 2 such as a Read Only Memory (ROM), a Random Access Memory (RAM), or a flash memory, or it may be stored in a detachably-attached storage medium (a non-transitory storage medium) such as a memory card and be installed in a storage device by mounting the storage medium in the driving monitoring device 1. The program (software) may be downloaded in advance from another computer device through short-range communication or wide-area communication by an application executed on the first terminal device T1 and transmitted from the first terminal device T1, whereby it may be installed in the storage device.

In the storage 70, for example, amount-of-change information 72, programs (for example, the programs described above), various kinds of other information, and the like are stored. The storage 70 may be realized by various kinds of storage devices described above, an Electrically Erasable Programmable Read Only Memory (EEPROM), or the like. The communicator 60 is an interface for performing wireless communication or wired communication with an information processing device (external device) such as the first terminal device T1.

The driving monitoring device 1 (the information processing device 2) performs at least an oversight warning process and an abnormality notification process. The oversight warning process is a process of detecting a driver's oversight of a risk object on the basis of the surrounding image and the face image and causing the warning part 82 to issue a warning. The abnormality notification process is a process of detecting a driver's abnormality (a decline in the cognitive ability) based on the surrounding image and the face image and causing the notification part 84 to issue a notification.

A risk object is, for example, an object that the vehicle M should avoid. A risk object is, for example, an object that interferes or has a likelihood of interfering when the vehicle M moves in its current state. An object having a likelihood of interfering is, for example, an object for which the degree of proximity becomes equal to or greater than a predetermined degree after a predetermined time when the position, the moving direction, and the moving speed of the object and the position, the moving method, and the moving speed of the vehicle M are taken into account. The object may be a traffic participant such as a pedestrian, a bicycle, or another vehicle or may be an object other than a traffic participant such as an object placed on a road or fallen debris.

### [Oversight Warning Process]

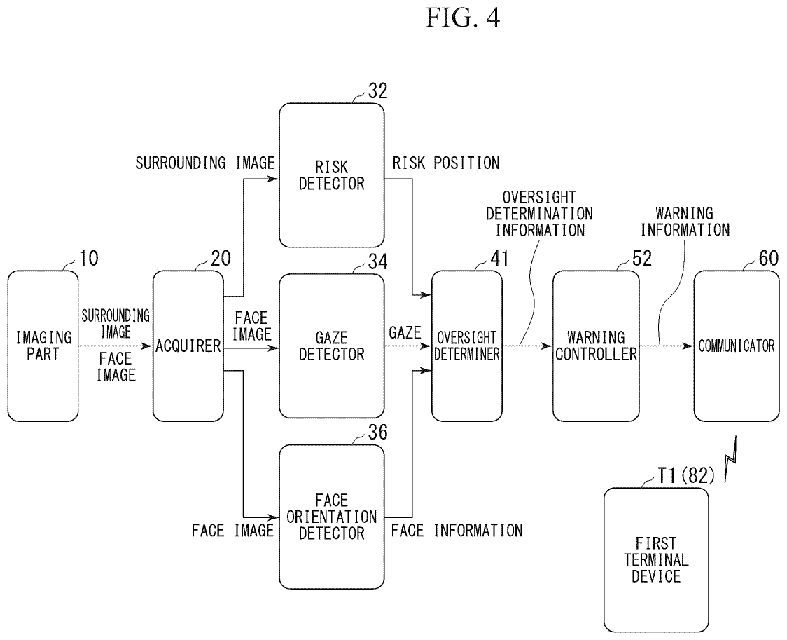

FIG. 4 is a diagram for describing an oversight warning process performed by the driving monitoring device 1 (the information processing device 2).

The acquisitor 20 acquires a front image and a rear image from the imaging part 10. Thus, the acquisitor 20 acquires surrounding images and face images in association with each other. Since the imaging part 10 repeatedly (periodically) captures a surrounding image and a face image, the acquisitor 20 repeatedly (periodically) acquires the surrounding image and the face image. The acquisitor 20 may acquire the surrounding image and the face image in association with the time at which they were captured (imaging time).

The risk detector 32 detects a risk object in the surrounding image. “Detecting a risk object” means, for example, detecting the existence of a risk object and the position of the risk object (hereinafter referred to as a risk position) relative to the vehicle M (the driving monitoring device 1). For example, reference information (not shown) representing a relation between the risk position in the surrounding image and the relative position with respect to the vehicle M may be stored in the storage 70 in advance. The risk detector 32 may detect the position of the risk object relative to the vehicle M on the basis of this reference information.

By detecting the relative position of the risk object with respect to the vehicle M, the risk detector 32 also detects the direction of existence (approach direction) of the risk object relative to the vehicle M. The “direction of existence of the risk object” means, for example, the direction in which the risk object approaching the vehicle M (the driving monitoring device 1) exists relative to the vehicle M. Hereinafter, the “direction of existence of the risk object” may be referred to as a “risk existence direction”.

The gaze detector 34 detects the driver's gaze in the face image. More specifically, the gaze detector 34 detects the driver's gaze by analyzing the face image using a predetermined image analysis technique. “Detecting the gaze” means, for example, detecting the position of the driver's eyeballs and the direction of the gaze (the direction of the eyeballs).

The face orientation detector 36 detects at least the orientation of the driver's face in the face image. More specifically, the face orientation detector 36 detects the orientation of the driver's face by analyzing the face image using a predetermined image analysis technique. The face orientation detector 36 may detect the position of the driver's face in addition to the orientation of the driver's face. Hereinafter, information that combines the “orientation of the face” and the “position of the face” may be referred to as “face information.” The face orientation detector 36 detects the face information in the face image.

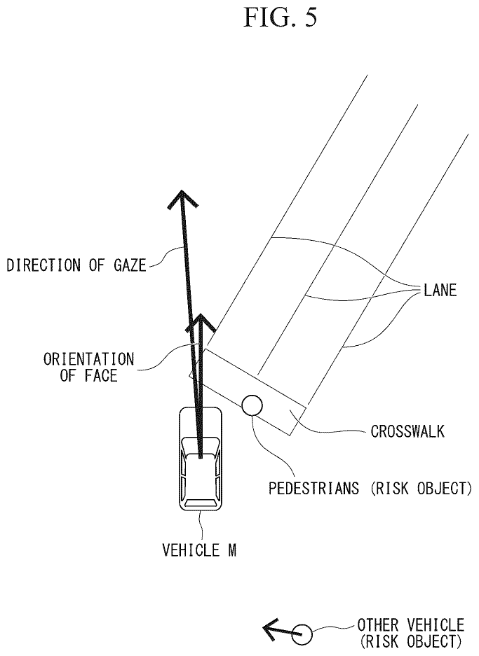

The detector 30 may be configured to detect lanes, crosswalks, and the like in addition to the risk objects, the gaze, and the face information described above. FIG. 5 is a diagram showing an example of information detected by the detector 30. In the example of FIG. 5, pedestrians, other vehicles, lanes, and crosswalks detected by the detector 30 are shown with reference to the vehicle M, the direction of the gaze, and the orientation of the face. Pedestrians and other vehicles are examples of traffic participants (objects) that can become risk objects. The detected information may be traffic participants (objects) that can be risk objects detected by the detector 30 represented with reference to the vehicle M, the orientation of the vehicle M, and/or the traveling direction of the vehicle M instead of the vehicle M, the direction of the gaze, and the orientation of the face.

The oversight determiner 41 (see FIG. 4) determines whether or not the driver has overlooked a risk object on the basis of the risk position and/or the risk existence direction, the gaze, and the face information. Each of the “risk position” and the “risk existence direction” is an example of “information relating to the position of a risk object.” The gaze and the face information are examples of “information relating to a face image.”

For example, reference information (not shown) representing a relation between the direction of the driver's eyeballs (gaze) and/or the orientation of the face in the face image and a relative direction with respect to the traveling direction of the vehicle M may be stored in the storage 70 in advance. The oversight determiner 41 may refer to this reference information to determine whether or not the direction of the gaze and/or the orientation of the face match the risk existence direction. In a case in which the direction of the gaze and/or the orientation of the face match the risk existence direction, the oversight determiner 41 may determine that the driver visually recognizes the risk object. “The case in which the direction of the gaze and/or the orientation of the face match the risk existence direction” may be, for example, a case in which the risk position exists on a straight line extending along the direction of the gaze and/or the orientation of the face. Alternatively, “the case in which the direction of the gaze and/or the orientation of the face match the risk existence direction” may be a case in which the risk position exists within a conical region having the viewpoint (eyeballs) and/or the position of the face as vertexes, having a central axis extending along the direction of the gaze and/or the orientation of the face, and expanding laterally as it extends forward. In a case in which the direction of the gaze and/or the orientation of the face do not match the position of the risk object, the oversight determiner 41 may determine that the driver does not visually recognize the risk object.

In a case in which a state in which the driver does not visually recognize the risk object continues for a predetermined time or longer, the oversight determiner 41 may determine that the driver has overlooked the risk object. In a case in which the driver visually recognizes the risk object or in a case in which the state in which the driver does not visually recognize the risk object is resolved before a predetermined time elapses, the oversight determiner 41 may determine that the driver has not overlooked the risk object. However, the technique for determining an oversight using the oversight determiner 41 is not limited to that described above and may be changed as appropriate.

The oversight determiner 41 generates oversight determination information on the basis of the determination result described above. The oversight determination information includes, for example, the presence/absence of the oversight described above and the risk existence direction.

The warning controller 52 causes the first terminal device T1 (the warning part 82) to output a warning on the basis of the oversight determination information. For example, the warning controller 52 transmits warning information to the first terminal device T1 through the communicator 60. The warning information includes, for example, information representing necessity of a warning and the risk existence direction. For example, in a case in which the oversight determination information includes information indicating that “there is an oversight,” the warning controller 52 generates warning information including information representing that “a warning is required.” For example, in a case in which the oversight determination information includes information indicating that “there is no oversight,” the warning controller 52 generates warning information including information representing that “a warning is not required.” The first terminal device T1 outputs a warning on the basis of the warning information.

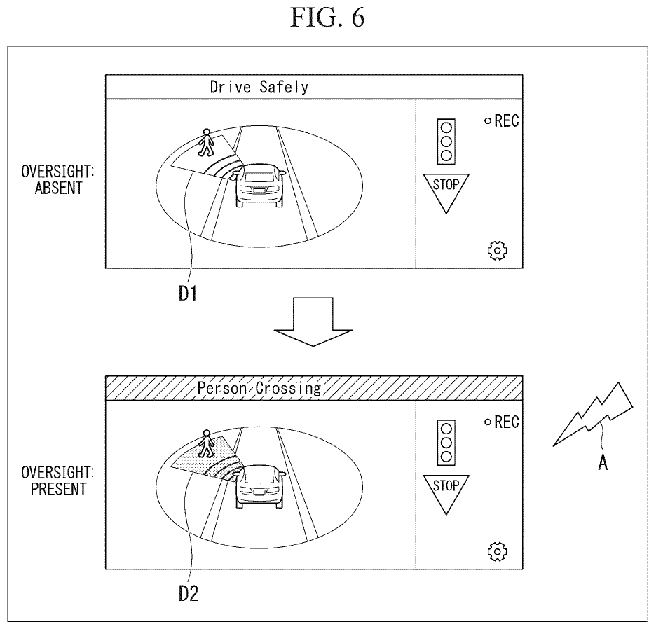

FIG. 6 is a diagram showing an example of a warning issued by the first terminal device T1 (a warning part 82). In the example of FIG. 6, the first terminal device T1 displays content that allows risk existence directions (D1 and D2 in the drawing) to be recognized. The first terminal device T1 issues a warning by changing the display form of the risk existence direction between a case where there is an oversight of the risk object (that is, a case in which the warning information includes information indicating that “a warning is required”) and a case in which there is no oversight of the risk object (that is, a case in which the warning information includes information indicating that “a warning is not required”). More specifically, in a case in which there is an oversight of the risk object, the risk existence direction is displayed in a more emphasized form than in a case in which there is no oversight. The emphasized form, for example, may be any form that allows the driver to more easily visually recognize the risk existence direction such as emphasis by color or emphasis by blinking or lighting icons or the like. In addition to or instead of the description presented above, the first terminal device T1 may issue a warning using sound output from a speaker, light emission from a light, or the like (A in the drawing). As long as the warning can be recognized by the driver, a specific method of the warning may be changed as appropriate.

The warning using the first terminal device T1 (the warning part 82) may be issued, for example, at a timing during the driver's driving of the vehicle M (that is, in real time). Examples of the “timing during the driver's driving of the vehicle M” include a timing during the traveling of the vehicle M, a timing during temporary stop of the vehicle M, and the like. For example, the transmission of the warning information performed by the warning controller 52 may be performed at the timings described above. Alternatively, the transmission of the warning information performed by the warning controller 52 may be performed at an arbitrary timing, and the first terminal device T1 (the warning part 82) may control the timing of the warning such that the warning is issued at the timings described above.

### [Abnormality Notification Process]

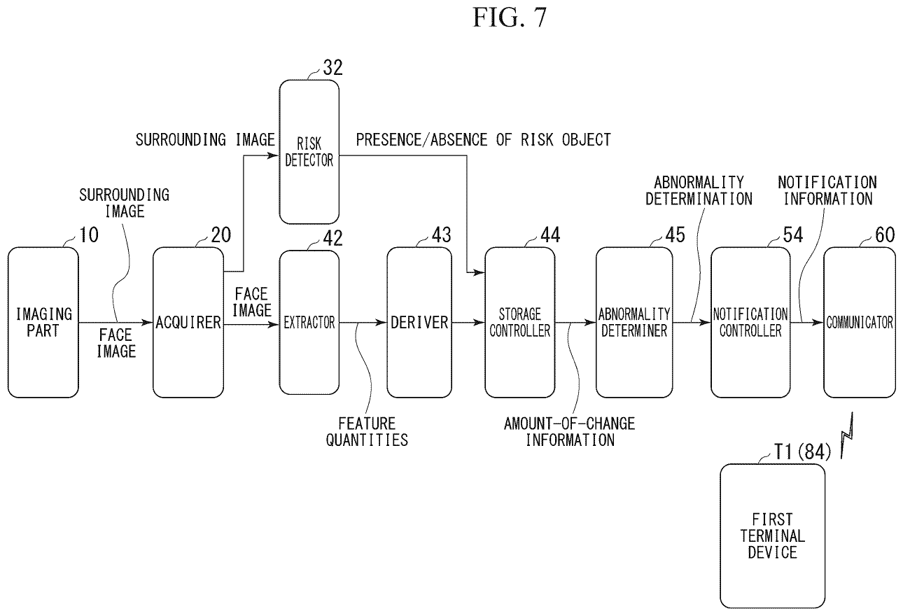

FIG. 7 is a diagram for describing an abnormality notification process performed by the driving monitoring device 1 (the information processing device 2).

Similarly to the oversight warning process described above, the acquisitor 20 acquires a front image and a rear image from the imaging part 10. In accordance with this, the acquisitor 20 acquires a surrounding image and a face image in association with each other. Since the imaging part 10 captures surrounding images and face images in a time series, the acquisitor 20 acquires the surrounding images and the face images in a time series. The acquisitor 20 may acquire the surrounding image and the face image in association with the times at which they were captured (imaging times).

The extractor 42 extracts feature quantities of the face of the driver in the face images acquired in time series. The extractor 42 may extract feature quantities of the face of the driver from each of a plurality of face images acquired in a time series. For example, the extractor 42 may extract feature quantities of the face of the driver by analyzing the face images using a predetermined image analysis technique. The feature quantities extracted by the extractor 42 may be changed on the basis of the facial expression of the driver.

The deriver 43 derives a change tendency of the feature quantities. More specifically, the deriver 43 derives an amount of change between a first feature quantity and a second feature quantity. The first feature quantity is extracted from a face image acquired at a first time point. The second feature quantity is extracted from a face image acquired at a second time point earlier than the first time point. The amount of change between the first feature quantity and the second feature quantity is, for example, an absolute value of a difference between the first feature quantity and the second feature quantity. The amount of change may be any value based on a result of comparison between the first feature quantity and the second feature quantity. The first time point and the second time point may be two time points that are consecutive in time.

The risk detector 32 detects a risk object in the surrounding images in the same manner as in the oversight warning process described above. In other words, the risk detector 32 determines whether or not a risk object is present. In other words, the risk detector 32 detects presence or absence of a risk object in the surrounding image.

The storage controller 44 causes the storage 70 to store a derivation time, the amount of change, and the presence or absence of a risk object in association with each other. The derivation time is a time at which the amount of change is derived. The derivation time may be acquired, for example, from information of an imaging time acquired in association with a face image. For example, the storage controller 44 causes the storage 70 to store the presence or absence of a risk object in a surrounding image acquired at the same time as the derivation time in association with the derivation time and the amount of change. The above-described process performed by the storage controller 44 is performed, for example, when the amount of change is derived by the deriver 43. Hereinafter, the information stored in the storage 70 in this way is referred to as amount-of-change information 72.

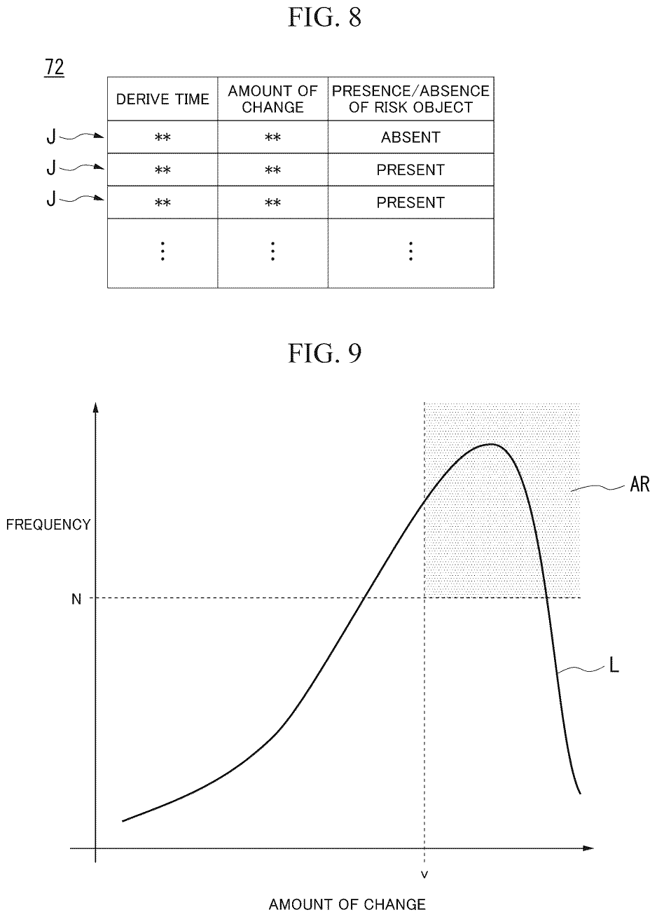

FIG. 8 is a diagram showing an example of the amount-of-change information 72. By repeating the above-described process using the storage controller 44, a plurality of sets of information each including the derivation time, the amount of change, and the presence or absence of a risk object (hereinafter referred to as a derivation information set J) are accumulated in the amount-of-change information 72. In other words, the storage controller 44 generates a derivation information set J each time the amount of change is derived by the deriver 43. Then, the storage controller 44 updates the amount-of-change information 72 by adding the generated derivation information set J to the amount-of-change information 72.

The abnormality determiner 45 determines whether or not an abnormality has occurred in the driver on the basis of the amount-of-change information 72.

First, the abnormality determiner 45 determines whether or not an amount of change derived on the basis of feature quantities extracted at each of a first time point and a second time point that are consecutive in time is a first threshold set in advance or more. The first threshold may be set in advance or may be set on the basis of a change tendency of a plurality of feature quantities extracted in the past from a plurality of face images of the driver. For example, the first threshold may be set on the basis of the amount of change derived when it is determined that no abnormality has occurred in the driver (the cognitive ability has not declined).

Next, the abnormality determiner 45 determines whether or not an abnormality has occurred in the driver on the basis of the number of times that the amount of change is the first threshold or more within a predetermined period. The predetermined period may be, for example, one driving session, or a defined time period. One driving session, for example, refers to driving in a period from the start to the end of the engine. The defined time period, for example, represents a fixed period such as 10 minutes or 1 hour. The abnormality determiner 45 determines that an abnormality has occurred in the driver when the number of times within the predetermined period that the amount of change is the first threshold or more is a set number or more. The abnormality determiner 45 determines that no abnormality has occurred in the driver in a case in which the number of times within the predetermined period that the amount of change is the first threshold or more is less than the set number. The set number, for example, may be set on the basis of the length of a predetermined period or may be set on the basis of the number of times that the amount of change was the first threshold or more in the past. For example, the set number may be set on the basis of the number of times that the amount of change was the first threshold or more when it was determined that no abnormality had occurred in the driver (that is, when the cognitive ability had not declined).

FIG. 9 is a diagram showing an example of a relationship between the amount of change and a frequency. In a graph of FIG. 9, the vertical axis represents the frequency, and the horizontal axis represents the amount of change. The vertical axis has a positive direction upward, and the frequency increases toward the upward direction. The horizontal axis has a positive direction toward the right side, and the amount of change increases toward the right side. When the driver's facial expressions at the first time point and the second time point are compared with each other, the amount of change increases as the change in the facial expression becomes larger, and it decreases as the change in the facial expression becomes smaller. The amount of change may be represented by a numerical value or may be represented by levels such as high, medium, and low.

FIG. 9 includes an amount-of-change index L. The amount-of-change index L is information that represents a correlation between the magnitude of the amount of change and the frequency in driving over a predetermined period. The abnormality determiner 45 calculates the frequency by counting the number of occurrences of each amount of change on the basis of a plurality of derivation information sets J stored during the predetermined period and derives the amount-of-change index L. In other words, the frequency represents the number of times that each amount of change has been derived.

The abnormality determiner 45 determines whether or not some or all of the amount-of-change indexes L exist in an abnormality determination region AR. The abnormality determination region AR is a region in the graph in which the amount of change is the first threshold V or more, and the frequency is the set number N or more. In other words, in a case in which some or all of the amount-of-change index L exists in the abnormality determination region AR, the number of times that the amount of change is the first threshold V or more is the set number N or more. In a case in which all of the amount-of-change indexes L do not exist in the abnormality determination region AR, the number of times that the amount of change is the first threshold V or more is less than the set number N. In other words, the abnormality determiner 45 determines that an abnormality has occurred in the driver in a case in which some or all of the amount-of-change indexes L exist in the abnormality determination region AR. The abnormality determiner 45 determines that no abnormality has occurred in the driver in a case in which all of the amount-of-change indexes L do not exist in the abnormality determination region AR.

The above-described process (a first determination process) performed by the abnormality determiner 45 may be performed, for example, at a timing other than during the driver's driving of the vehicle M. Examples of “a timing other than during the driver's driving of the vehicle M” include timings when the vehicle M is stopped, after the driver has ended driving the vehicle M, when the driver has left the vehicle M and is at home or the like, and the like. In a case in which the first determination process is performed at a timing other than during the driver's driving of the vehicle M, the first determination process is executed for each predetermined period for a plurality of derivation information sets J stored during a period after a date on which the previous first determination process was performed. The first determination process using the abnormality determiner 45 may be performed during the driver's driving of the vehicle M every time a predetermined period elapses. In a case in which the first determination process is performed every time the predetermined period elapses, the derivation information sets J included in the amount-of-change information 72 may be deleted after the end of the first determination process. In this case, the abnormality determiner 45 performs the first determination process using all the derivation information sets J included in the amount-of-change information 72.

In this way, by determining whether or not an abnormality has occurred in the driver on the basis of the number of times that the amount of change is the first threshold or more, a decline in the driver's cognitive ability can be detected. That the amount of change is the first threshold or more means that the change in the facial expression is large. In a case in which the number of times that the amount of change is the first threshold or more is large, it is predicted that the number of times of rapid emotional changes is larger, that is, emotional fluctuations are intense. Patients who have developed dementia tend to exhibit intense emotional fluctuations. In other words, by performing determination based on the change tendency of feature quantities, a decline in the driver's cognitive ability can be detected.

The abnormality determiner 45 may determine whether or not an abnormality has occurred in the driver on the basis of the presence or absence of a risk object and the amount of change. On the basis of the derivation information set J, in a case in which it is determined that no risk object is present, and the amount of change is a second threshold or more, the abnormality determiner 45 determines that an abnormality has occurred in the driver. On the basis of the derivation information set J, in a case in which it is determined that no risk object is present, and the amount of change is less than the second threshold, the abnormality determiner 45 determines that no abnormality has occurred in the driver. On the basis of the derivation information set J, in a case in which it is determined that a risk object is present, and the amount of change is less than a third threshold, the abnormality determiner 45 determines that an abnormality has occurred in the driver. On the basis of the derivation information set J, in a case in which it is determined that a risk object is present, and the amount of change is the third threshold or more, the abnormality determiner 45 determines that no abnormality has occurred in the driver. The second threshold and the third threshold may be set in advance or may be set on the basis of a change tendency of a plurality of feature quantities extracted in the past from face images of the driver. The second threshold, for example, has a value that is larger than the first threshold. The third threshold, for example, has a value that is smaller than the first threshold. The second threshold and the third threshold may have the same value or different values. The second threshold and/or the third threshold may have the same value as the first threshold or values different from the first threshold.

The above-described process (a second determination process) performed by the abnormality determiner 45, for example, may be performed at a timing other than during the driver's driving of the vehicle M. Examples of the “timing other than during the driver's driving of the vehicle M” include timings when the vehicle M is stopped, after the driver has ended the driving the vehicle M, and when the driver has left the vehicle M and is at home, and the like. In a case in which the process is performed at a timing other than during the driver's driving of the vehicle M, the abnormality determiner 45 performs the second determination process for each of all the plurality of derivation information sets J stored after a time at which the previous second determination process was performed. The second determination process performed by the abnormality determiner 45 may be performed every time the amount of change is derived. In a case in which the second determination process is performed every time the amount of change is derived, the abnormality determiner 45 refers to the latest derivation information set J included in the amount-of-change information 72 and performs the second determination.

Patients who have developed dementia tend to suffer from disorientation. The disorientation refers to a decline in the recognition ability for his or her situation. For example, in a case in which the driver's facial expression changes significantly in a state in which no risk object is detected (the degree of risk is low) or in a case in which the driver's facial expression changes insufficiently in a state in which a risk object is detected (the degree of risk is high), the driver's cognitive ability is reduced, that is, the driver has a likelihood of having developed dementia. In this way, by determining whether or not an abnormality has occurred in the driver on the basis of the presence or absence of a risk object and the amount of change, a decline in the driver's cognitive ability can be appropriately detected.

However, the technique for determining whether or not an abnormality has occurred in the driver using the abnormality determiner 45 is not limited to that described above and can be changed appropriately.

The abnormality determiner 45 generates abnormality determination information on the basis of a result of the determination. The abnormality determination information includes, for example, the presence/absence of occurrence of an abnormality in the driver described above and the amount-of-change index. In a case in which the first determination process and the second determination process are executed at different timings, the abnormality determiner 45 may generate abnormality determination information on the basis of results of the respective determinations. At this time, the abnormality determination information may include the presence/absence of occurrence of an abnormality in the driver, the amount-of-change index, and a type of determination process. The abnormality determination information may be stored in the storage 70.

The notification controller 54 causes the first terminal device T1 (the notification part 84) to output a notification on the basis of the abnormality determination information. For example, the notification controller 54 transmits notification information to the first terminal device T1 through the communicator 60. For example, in a case in which the abnormality determination information includes information indicating that an abnormality has occurred in the driver (that is, in a case in which the abnormality determiner 45 determines that an abnormality has occurred in the driver), the notification controller 54 may transmit first notification information to the first terminal device T1. The first notification information includes, for example, information representing driving evaluation information. In a case in which the abnormality determination information includes information indicating that no abnormality has occurred in the driver (that is, in a case in which the abnormality determiner 45 determines that no abnormality has occurred in the driver), the notification controller 54 may not transmit the first notification information to the first terminal device T1.

The first terminal device T1 (the notification part 84) outputs a notification on the basis of the first notification information. The notification may be given, for example, by causing a display device included in the first terminal device T1 (the notification part 84) to display text, images, or the like based on the first notification information.

The notification given by the first terminal device T1 (the notification part 84) may include content recommending to receive medical treatment from a medical institution (for example, the driver's primary care physician). In this case, the first terminal device T1 (the notification part 84) may cause the driver to input information indicating whether or not the driver accepts the reception of medical treatment from the medical institution. More specifically, a display device included in the first terminal device T1 (the notification part 84) may be caused to display a virtual button or the like that indicates the acceptance of reception of medical treatment. Such a button or the like functions as an acceptance information acquisitor that acquires information indicating the driver's intention to accept the reception of medical treatment from the medical institution (hereinafter referred to as acceptance information). In a case in which the driver operates the first terminal device T1 (the notification part 84), and the acceptance information acquisitor acquires the acceptance information, the notification controller 54 or the first terminal device T1 (the notification part 84) may cause a predetermined information processing terminal to give a notification representing that the driver has accepted the reception of medical treatment. The predetermined information processing terminal may be, for example, a terminal used in a medical institution (for example, a second terminal device T2 described below). More specifically, the predetermined information processing terminal may be a terminal that is used by personnel belonging to the medical institution (for example, a doctor, a nurse, or the like).

However, as long as the notification is given in a form that can be recognized by the driver, the form of the notification given by the first terminal device T1 (the notification part 84) is not particularly limited and may be appropriately modified.

The notification given by the first terminal device T1 (the notification part 84) may be given, for example, at a timing other than during the driver's driving of the vehicle M. Examples of the “timing other than during the driver's driving of the vehicle M” include timings when the vehicle M is stopped, after the driver has ended driving the vehicle M, when the driver has left the vehicle M and is at home or the like, and the like. For example, the transmission of the first notification information from the notification controller 54 may be performed at the timing described above. Alternatively, the transmission of the first notification information from the notification controller 54 may be performed at an arbitrary timing, and the first terminal device T1 (the notification part 84) may control the timing of the notification such that the notification is given at the above-described timing.

### [Process Flow]

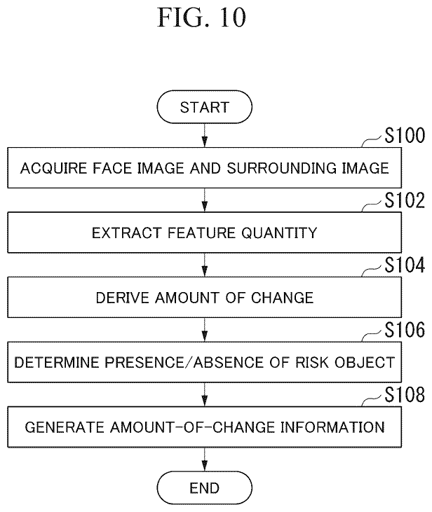

FIG. 10 is a flowchart showing an example of the flow of a process executed by the information processing device 2. The process of the flowchart shown in FIG. 10 is started, for example, while the vehicle M is traveling.

First, the acquisitor 20 acquires a face image and a surrounding image (Step S100). Next, the extractor 42 extracts feature quantities of the face of the driver on the basis of the face image (Step S102). Next, the deriver 43 derives the amount of change of the feature quantities (Step S104). More specifically, the deriver 43 derives the amount of change between a first feature quantity extracted from a face image acquired at a first time point and a second feature quantity extracted from a face image acquired at a second time point that is earlier than the first time point. Next, the risk detector 32 detects a risk object (Step S106). In other words, the risk detector 32 determines the presence or absence of a risk object. Next, the storage controller 44 generates amount-of-change information 72 (Step S108). The amount-of-change information 72 includes a derivation time, the amount of change, and the presence/absence of a risk object. Thereafter, the information processing device 2 ends the process shown in FIG. 10.

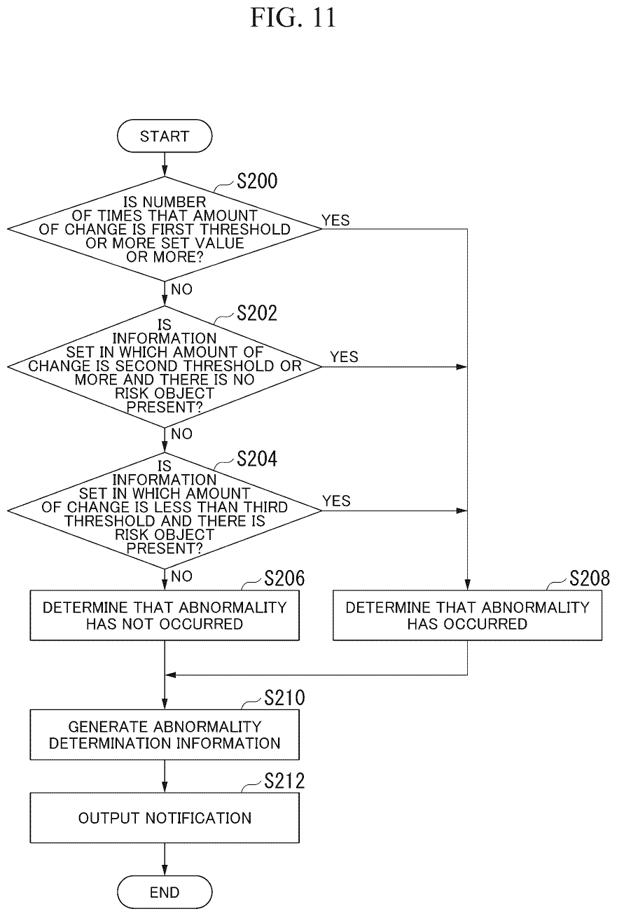

The order of the processes described above may be changed. For example, the process of Step S106 may be executed before the process of Step S100. The same applies to the flowchart shown in FIG. 11 described below.

FIG. 11 is a diagram showing an example of the flow of a process executed by the information processing device 2. The process of the flowchart shown in FIG. 11 may be repeatedly started, for example, at a predetermined timing such as after completion of driving or at a predetermined cycle, or may be started in accordance with an instruction from the driver or the like.

First, the abnormality determiner 45 refers to the amount-of-change information 72 and determines whether or not the number of times the amount of change is a first threshold or more within a predetermined period is a set number or more (Step S200). In a case in which the number of times the amount of change is the first threshold or more is less than the set number within the predetermined period (Step S200; No), the abnormality determiner 45 determines whether or not a derivation information set J in which the amount of change is a second threshold or more, and no risk object is determined to be present is stored (Step S202). In a case in which no derivation information set J in which the amount of change is the second threshold or more, and no risk object is determined to be present is not stored (Step S202; No), the abnormality determiner 45 determines whether or not a derivation information set J in which the amount of change is less than a third threshold, and a risk object is determined to be present is stored (Step S204). In a case in which a derivation information set J in which the amount of change is less than the third threshold, and a risk object is determined to be present is not stored (Step S204; No), the abnormality determiner 45 determines that no abnormality has occurred in the driver (Step S206).

On the other hand, in a case in which the number of times the amount of change is the first threshold or more is the set number or more within the predetermined period (Step S200; Yes), in a case in which a derivation information set J in which the amount of change is the second threshold or more, and no risk object is determined to be present is stored (Step S202; Yes), or in a case in which a derivation information set J in which the amount of change is less than the third threshold, and a risk object is determined to be present is stored (Step S204; YES), the abnormality determiner 45 determines that an abnormality has occurred in the driver (Step S208).

After the end of the process of Step S206 or after the end of the process of Step S208, the abnormality determiner 45 generates abnormality determination information (Step S210). Next, the notification controller 54 causes the first terminal device T1 (the notification part 84) to output a notification on the basis of the abnormality determination information (Step S212). Thereafter, the information processing device 2 ends the process of the flowchart shown in FIG. 11.

The processes of Steps S200 to S210 described above and the process of Step S212 may be executed at different timings. For example, the processes of Steps S200 to S210 are executed for every predetermined period, and the generated abnormality determination information is stored in the storage 70. Thereafter, at a timing designated by the driver, the abnormality determiner 45 may refer to the abnormality determination information and execute the process of Step S212.

According to the first embodiment described above, the information processing device 2 includes an acquisitor that acquires face images, which are images including a face of a driver of a vehicle, in a time series, an extractor that extracts feature quantities of the face of the driver from each of the face images, and an abnormality determiner that determines whether or not an abnormality has occurred in the driver on the basis of a change tendency of the feature quantities. In accordance with this, a decline in the driver's cognitive ability can be detected. For example, by giving a notification of information relating to a decline in cognitive ability to an elderly driver in an early stage, accidents can be prevented in advance. The driving lifespan is extended, and a safe and secure driving experience can be provided, whereby the creation of a rich life through extension of healthy life expectancy can be enabled.

### Second Embodiment

Next, a second embodiment is described, and the basic configuration thereof is similar to that of the first embodiment. For this reason, the same reference signs are assigned to the same components, and description thereof is omitted, and only differences are described.

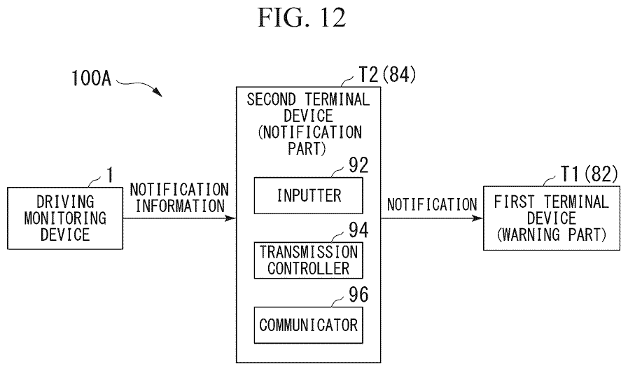

FIG. 12 is a diagram for describing an abnormality notification process of an information processing system 100A according to the second embodiment. As shown in FIG. 12, the information processing system 100A according to the second embodiment includes a second terminal device T2 in addition to a driving monitoring device 1 and a first terminal device T1.

The second terminal device T2 is an information processing device used by a user (for example, personnel of a hospital or an insurance company) different from the driver using the first terminal device T1. The second terminal device T2 may be, for example, a smartphone, a tablet terminal, a personal computer, or the like.

In the second embodiment, the driving monitoring device 1 (a notification controller 54) transmits first notification information to not the first terminal device T1 but the second terminal device T2. In other words, in the second embodiment, the notification part 84 of which a notification is controlled by the notification controller 54 is not the first terminal device T1 but the second terminal device T2. In the second terminal device T2, for example, an application for receiving notifications from the information processing device 2 is executed. The application causes a display device to display images, character strings, or the like based on information (the first notification information or the like) transmitted from the information processing device 2 or causes a speaker to output a sound.

On the basis of information input by a user's operation, the second terminal device T2 causes a notification to be given to the first terminal device T1. The second terminal device T2 includes, for example, an inputter 92, a transmission controller 94, and a communicator 96.

Each of the inputter 92 and the transmission controller 94, for example, includes a hardware processor such as a central processing unit (CPU) and a storage device (a storage device including a non-transitory storage medium) storing a program (software), and the function of each constituent element is realized by the processor executing the program. Some or all of such constituent elements may be realized by hardware (including circuitry) such as an LSI, an ASIC, an FPGA, or a GPU, or the function of each constituent element may be realized by software and hardware in cooperation. For some or all of such constituent elements, the function of each of the constituent elements may be realized by a dedicated LSI. The communicator 96 is an interface for performing wireless communication or wired communication with an information processing device such as the first terminal device T1.

The inputter 92 receives a user's operation and inputs it to the second terminal device T2. The inputter 92 is configured using existing input devices such as a keyboard, a pointing device (a mouse, a tablet, and the like), buttons, and a touch panel. The inputter 92 may be an interface for connecting an input device to the second terminal device T2. In this case, the inputter 92 inputs an input signal generated in accordance with a user's operation by the input device to the second terminal device T2. The inputter 92 may be arbitrarily configured as long as it is capable of inputting instructions based on user's operations to the second terminal device T2.

For example, the user checks the content of a notification given to the second terminal device T2 by the driving monitoring device 1 (the notification controller 54) and receives content to be notified to the driver. The content to be notified to the driver includes, for example, a possibility of a decline in the cognitive ability and a recommendation to receive a medical treatment from a medical institution for the purpose of detecting a decline in the cognitive ability. The user inputs information including the reviewed notification content to the second terminal device T2 by operating the inputter 92. The transmission controller 94 transmits the information input by the user's operation to the first terminal device T1 through the communicator 96. The first terminal device T1 gives a notification to the driver on the basis of the transmitted information.

The information processing system 100A may be configured such that the driver can restrict transmission of the first notification information from the driving monitoring device 1 (the notification controller 54) to the second terminal device T2 by operating the first terminal device T1 or the driving monitoring device 1. For example, by the driver operating the first terminal device T1 or the driving monitoring device 1, restriction information indicating whether transmission of the first notification information described above is restricted is generated, and the generated restriction information may be stored in the storage 70. Then, the warning controller 52 may determine whether to transmit the first notification information to the second terminal device T2 on the basis of the restriction information stored in the storage 70. According to such a configuration, it is determined in accordance with the driver's intention whether to notify the user using the second terminal device T2 of information relating to an abnormality of the driver. Therefore, the driver's privacy can be more easily protected.

According to the second embodiment described above, the information processing system 100A includes the information processing device 2, the first terminal device T1 that is used by the driver and is not the notification part 84 of which a notification is controlled by the notification controller 54, and the second terminal device T2 that is used by a user different from the driver and is the notification part 84 of which a notification is controlled by the notification controller 54, and the second terminal device T2 causes a notification to be given to the first terminal device T1 on the basis of information input by a user's operation. In accordance with this, notification content that has been reviewed by the user using the second terminal device T2 can be provided for the driver using the first terminal device T1. For example, in a case in which medical personnel use the second terminal device T2, an abnormality of the driver can be detected by using the abnormality determination information for medical determination.

### Modified Example

The technical scope of the present invention is not limited to the above-described embodiments, and various modifications may be made without departing from the spirit of the present invention.

For example, the abnormality determiner 45 may set the amount-of-change index L derived when it is determined that no abnormality has occurred in the driver (the cognitive ability has not declined) as a reference amount-of-change index L1 and determine whether an abnormality has occurred in the driver by comparing the reference amount-of-change index L1 with the amount-of-change index L. For example, the abnormality determiner 45 may determine that an abnormality has occurred in the driver in a case in which an absolute value of a difference between the amount of change corresponding to a mode of the reference amount-of-change index L1 and the amount of change corresponding to a mode of the amount of change index L is a threshold or more. For example, the abnormality determiner 45 may determine that no abnormality has occurred in the driver in a case in which an absolute value of a difference between the amount of change corresponding to a mode of the reference change amount index L1 and the amount of change corresponding to a mode of the amount-of-change index L is less than a threshold. The reference amount-of-change index L1 may be set on the basis of an average driving ability of a normal person.

For example, the driving monitoring device 1 (the information processing device 2) may perform another warning process (hereinafter referred to as an auxiliary warning process) in addition to (or in place of) the above-described oversight warning process. The auxiliary warning process may be, for example, a collision warning process in which a likelihood of a risk object colliding with the vehicle M is detected on the basis of a position and a relative speed of the risk object with respect to the vehicle M, and a warning is issued by the warning part 82. In the collision warning process, the relative speed of the risk object with respect to the vehicle M may be calculated, for example, by analyzing, a change in the risk position detected by the risk detector 32 over time using a processor 40. In a case in which the processor 40 detects a likelihood of the risk object colliding with the vehicle M, the warning controller 52 may cause the warning part 82 to issue a warning. Similar to the oversight warning process, the auxiliary warning process may be performed at a timing during the driver's driving of the vehicle M (that is, in real time). In a configuration in which the driving monitoring device 1 (the information processing device 2) performs the auxiliary warning process, a warning using the warning part 82 based on a result of the determination of overlooking performed by the oversight determiner 41 may not be issued. Even in this case, the determination of overlooking performed by the oversight determiner 41 is executed for the above-described abnormality notification process.

Information relating to results of the determination of overlooking performed by the oversight determiner 41 and the like may be accumulated in the storage 70, and content generated on the basis of the accumulated information for allowing the driver to review his or her own past driving (hereinafter referred to as “review content”) may be provided for the driver by the first terminal device T1 (the warning part 82). The review content may include, for example, information (such as text or images) that allows the driver to check risk objects that the driver overlooked. The review content may be generated by the driving monitoring device 1 (the information processing device 2), or may be generated by the first terminal device T1 (the warning part 82).

In the information processing system 100A according to the second embodiment, the driving monitoring device 1 (the notification controller 54) may transmit the first notification information to both the first terminal device T1 and the second terminal device T2. In other words, both the first terminal device T1 and the second terminal device T2 may be a notification part 84 of which notifications are controlled by the notification controller 54.

In the first embodiment, the driving monitoring device 1 (information processing device 2) and the first terminal device T1 are configured as separate devices; however, they may be configured as a single integrated device. For example, the driving monitoring device 1 may implement all of the functions of the imaging part 10, the acquisitor 20, the detector 30, the processor 40, the controller 50, the storage 70, the warning part 82, and the notification part 84. Alternatively, all of these functions may be implemented in the first terminal device T1 or in the vehicle M. Similarly, in the second embodiment, all of the functions of the imaging part 10, the acquisitor 20, the detector 30, the processor 40, the controller 50, the storage 70, and the warning part 82 may be implemented in the driving monitoring device 1, or all of these functions may be implemented in the first terminal device T1.

The driving monitoring device 1 may be implemented using a plurality of information processing devices. For example, the functions provided by the information processing device 2 may be implemented in the first terminal device T1 or the vehicle M (for example, an ECU). Alternatively, the driving monitoring device 1 may be implemented using a device such as a cloud. For example, in the driving monitoring device 1, the detector 30, the processor 40, the controller 50, and the storage 70 may be implemented in different information processing devices. For example, the storage 70 may be distributed and implemented across a plurality of information processing devices.

The embodiments and the modified example described above can be expressed as below.

An information processing device including: a storage medium storing computer-readable instructions; and a processor connected to the storage medium, the processor executing the computer-readable instructions to: acquire face images that are images including a face of a driver of a vehicle in a time series; extract feature quantities of the face of the driver in each of the face images; and determine whether or not an abnormality has occurred in the driver on the basis of a change tendency of the feature quantities.

The constituent elements according to the embodiments described above can be appropriately substituted with known constituent elements within a range not departing from the gist of the present invention, and the embodiments and the modified example described above may be appropriately combined together.

What is claimed is:

1. An information processing device comprising:
a storage medium storing computer-readable instructions; and
one or more processors connected to the storage medium,
the one or more processors executing the computer-readable instructions to:
acquire face images including a face of a driver of a vehicle in a time series;
extract feature quantities of the face of the driver on the basis of the face images; and
determine whether or not an abnormality has occurred in the driver on the basis of a change tendency of the feature quantities.

2. The information processing device according to claim 1, wherein the one or more processors execute the computer-readable instructions to
extract the feature quantities of the face of the driver in each of the face images acquired in the time series.

3. The information processing device according to claim 2, wherein the one or more processors execute the computer-readable instructions to:
derive an amount of change between a first feature quantity and a second feature quantity, the first feature quantity being extracted from the face image acquired at a first time point, the second feature quantity being extracted from the face image acquired at a second time point, the second time point being earlier than the first time point; and
determine whether or not an abnormality has occurred in the driver on the basis of the amount of change.

4. The information processing device according to claim 3, wherein the one or more processors execute the computer-readable instructions to:
calculate the amount of change between sets of two face images being consecutive in time in the face images acquired in the time series; and
determine whether or not an abnormality has occurred in the driver on the basis of the number of the amounts of change being a threshold or more among a plurality of the calculated amounts of change.

5. The information processing device according to claim 4, wherein the one or more processors execute the computer-readable instructions to calculate the amount of change for each of the sets of two consecutive face images.

6. The information processing device according to claim 3, wherein the one or more processors execute the computer-readable instructions to:
further acquire a surrounding image of the vehicle;
detect presence or absence of a risk object in the surrounding image; and
determine that an abnormality has occurred in the driver
in a case in which the risk object has not been detected, and
in a case in which the amount of change corresponding to a time point at which the surrounding image has been acquired is a threshold or more.

7. The information processing device according to claim 3, wherein the one or more processors execute the computer-readable instructions to:
further acquire a surrounding image of the vehicle;
detect presence or absence of a risk object in the surrounding image; and
determine that an abnormality has occurred in the driver
in a case in which the risk object has been detected, and
in a case in which the amount of change corresponding to a time point at which the surrounding image has been acquired is less than a threshold.

8. The information processing device according to claim 1, wherein the one or more processors execute the computer-readable instructions to
cause a notification part to give a notification
in a case in which it is determined that an abnormality has occurred in the driver.

9. An information processing system comprising:
the information processing device according to claim 1;
a first terminal device used by the driver; and
a second terminal device used in a medical institution,
the information processing device causing:
the first terminal device to perform content recommending to receive a medical treatment of the medical institution
in a case in which it is determined that an abnormality has occurred in the driver; and
the second terminal device to give a notification on the basis of information indicating an intention to accept the medical treatment input to the first terminal device by the driver.

10. An information processing system comprising:
the information processing device according to claim 1;
a first terminal device used by the driver; and
a second terminal device used by a user different from the driver,
the second terminal device having notifications controlled by the information processing device and
issuing a notification to the first terminal device on the basis of information input in accordance with a user's operation.

11. An information processing method using a computer, the information processing method comprising:
acquiring face images including a face of a driver of a vehicle in a time series;
extracting feature quantities of the face of the driver on the basis of the face images; and
determining whether or not an abnormality has occurred in the driver on the basis of a change tendency of the feature quantities.

12. A non-transitory storage medium having a program stored thereon that causes a computer to:
acquire face images including a face of a driver of a vehicle in a time series;
extract feature quantities of the face of the driver on the basis of the face images; and
determine whether or not an abnormality has occurred in the driver on the basis of a change tendency of the feature quantities.
